(19) United States
(12) Patent Application Publication
(10) Pub. No.: US 2024/0050074 A1
(43) Pub. Date: Feb. 15, 2024

(54) MEDICAL INFORMATION PROCESSING APPARATUS, A NON-TRANSITORY COMPUTER READABLE MEDIUM, AND ULTRASONIC DIAGNOSTIC APPARATUS

(71) Applicant: Canon Medical Systems Corporation, Otawara-shi (JP)

(72) Inventors: Mitsuo AKIYAMA, Otawara (JP); Minori IZUMI, Shioya (JP); Taku MURAMATSU, Otawara (JP); Katsuyuki TAKAMATSU, Yaita (JP); Tomoko AIZAWA, Nasushiobara (JP); Takashi KOYAKUMARU, Utsunomiya (JP)

(73) Assignee: Canon Medical Systems Corporation, Otawara-shi (JP)

(21) Appl. No.: 18/447,714

(22) Filed: Aug. 10, 2023

(30) Foreign Application Priority Data
Aug. 10, 2022 (JP) 2022-128095

(51) Int. Cl. A61B 8/08; A61B 8/14; G16H 30/40; G16H 30/20

(52) U.S. Cl. CPC A61B 8/5292; A61B 8/0883; A61B 8/14; A61B 8/5207; G16H 30/40; G16H 30/20

(57) ABSTRACT

According to one embodiment, a medical information processing apparatus includes processing circuitry. The processing circuitry determines whether or not a plurality of ultrasonic images are images collected by a stress echocardiography method. The processing circuitry adds attribute information to each of the ultrasonic images if it is determined that the ultrasonic images are images collected by the stress echocardiography method, the attribute information concerning at least one of a stress state of a subject and a slice of the ultrasonic image.

### CROSS-REFERENCE TO RELATED APPLICATIONS

This application is based upon and claims the benefit of priority from Japanese Patent Application No. 2022-128095, filed Aug. 10, 2022, the entire contents of which are incorporated herein by reference.

### FIELD

Embodiments described herein relate generally to a medical information processing apparatus, a non-transitory computer readable medium, and an ultrasonic diagnostic apparatus.

### BACKGROUND

In diagnosing ischemic heart disease, valvular disease, or the like, imaging is sometime performed by a stress echocardiography examination. Stress echocardiography is an imaging technique for diagnosing whether the cardiac function is normal or abnormal by comparatively observing an ultrasonic image before the application of stress to the heart and an ultrasonic image after the application of stress to the heart. In executing a stress echocardiography examination, it is desirable to execute an examination by using a stress echocardiography examination application upon carefully preparing equipment, a stress stage (stress level), slices to be collected, and the like in consideration of risks. The stress echocardiography examination application can classify ultrasonic images by tagging stress stages such as a rest state and a stress state to the ultrasonic images.

After an ordinary examination other than a stress echocardiography examination, in order to induce a symptom in a patient, a stress echocardiography examination is sometimes executed by the application of a simple stress such as a five-minute walk on a treadmill or hand gripping. In such a case, since no stress echocardiography application is used from the beginning, no tag information such as stress stages is attached to the ultrasonic images collected by an ordinary examination. Accordingly, a healthcare professional needs to determine in which stages ultrasonic images have been collected and to, for example, manually perform tagging and rearrange data.

### BRIEF DESCRIPTION OF THE DRAWINGS

FIG. 1 is a block diagram showing a data management system including a medical information processing apparatus according to this embodiment.

FIG. 2 is a block diagram showing a medical information processing apparatus according to this embodiment.

FIG. 3 is a block diagram showing an ultrasonic diagnostic apparatus according to this embodiment.

FIG. 4 is a flowchart showing an example of the operation of the medical information processing apparatus according to this embodiment.

FIG. 5 is a flowchart showing the details of attribute information addition processing by the medical information processing apparatus according to this embodiment.

FIG. 6 is a view showing an example of a GUI associating ultrasonic images with attribute information according to this embodiment.

FIG. 7 is a flowchart showing a case in which execution/non-execution of a stress echocardiography examination is determined in accordance with a user instruction.

### DETAILED DESCRIPTION

In general, according to one embodiment, a medical information processing apparatus includes processing circuitry. The processing circuitry determines whether or not a plurality of ultrasonic images are images collected by a stress echocardiography method. The processing circuitry adds attribute information to each of the ultrasonic images if it is determined that the ultrasonic images are images collected by the stress echocardiography method, the attribute information concerning at least one of a stress state of a subject and a slice of the ultrasonic image.

A medical information processing apparatus, a non-transitory computer readable medium, and an ultrasonic diagnostic apparatus according to this embodiment will be described below with reference to the accompanying drawings. In the following embodiments, portions denoted by the same reference numerals perform the same operations, and a redundant description will be appropriately omitted. An embodiment will be described below with reference to the accompanying drawings.

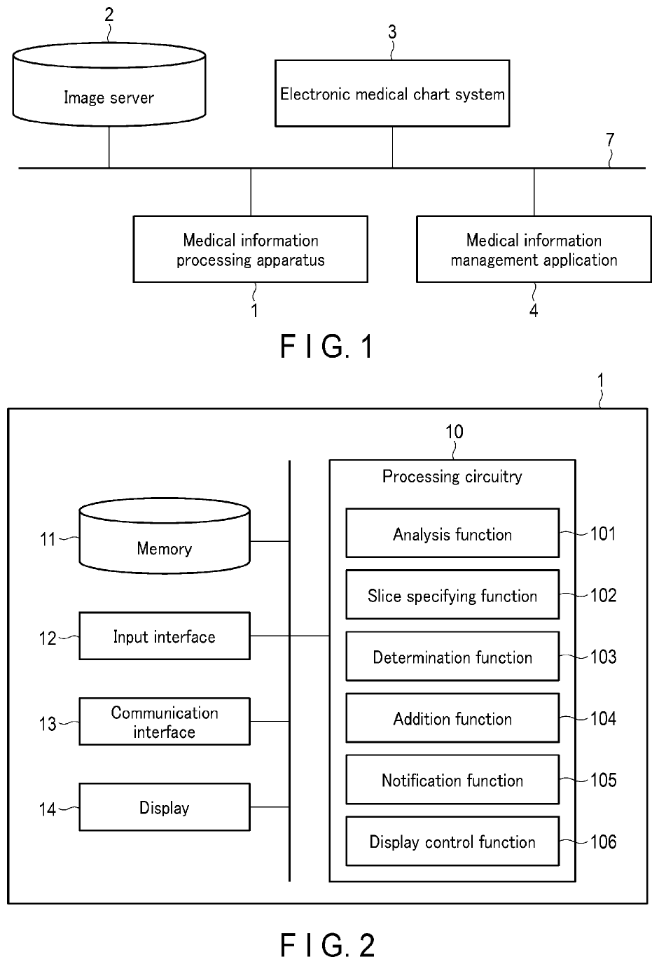

A data management system including the medical information processing apparatus according to this embodiment will be described with reference to FIG. 1.

The data management system shown in FIG. 1 includes a medical information processing apparatus 1, an image server 2, an electronic medical chart system 3, and a medical information management application 4. These components are connected via a network 7. Note that the network may be either a wired connection network or a wireless connection network. In addition, the line to be connected is not limited to an intra-hospital network as long as security is ensured. For example, each component may be connected to a public communication circuit such as the Internet via a VPN (Virtual Private Network).

The medical information processing apparatus 1 performs data management by adding attribute information such as an imaged slice or a stress state (stress stage or phase) to, for example, each ultrasonic image associated with a stress echocardiography examination.

The image server 2 is, for example, a PACS (Picture Archiving and Communication System), which is a system for storing medical image data such as ultrasonic images and managing the stored medical image data. The image server 2 stores and manages medical image data converted according to the DICOM (Digital Imaging and Communication Medicine) standard.

The electronic medical chart system 3 is a system that displays and manages patient information, medical treatment information, diagnosis reports, and the like.

The medical information management application 4 is a system that stores, for example, hospital information such as medical treatment information, patient information, and order information and manages the stored hospital information. This embodiment includes, for example, application software (to be referred to as a stress echocardiography application hereinafter) used for a stress echocardiography examination as the medical information management application 4.

The medical information processing apparatus 1 according to this embodiment will be described in detail next with reference to FIG. 2.

The medical information processing apparatus 1 includes processing circuitry 10, a memory 11, an input interface 12, a communication interface 13, and a display 14. The processing circuitry 10, the memory 11, the input interface 12, the communication interface 13, and the display 14 are communicably connected to each other via, for example, a bus.

The processing circuitry 10 is, for example, a processor such as a CPU (Central Processing Unit) or a GPU (Graphics Processing Unit), which includes an analysis function 101, a slice specifying function 102, a determination function 103, an addition function 104, a notification function 105, and a display control function 106.

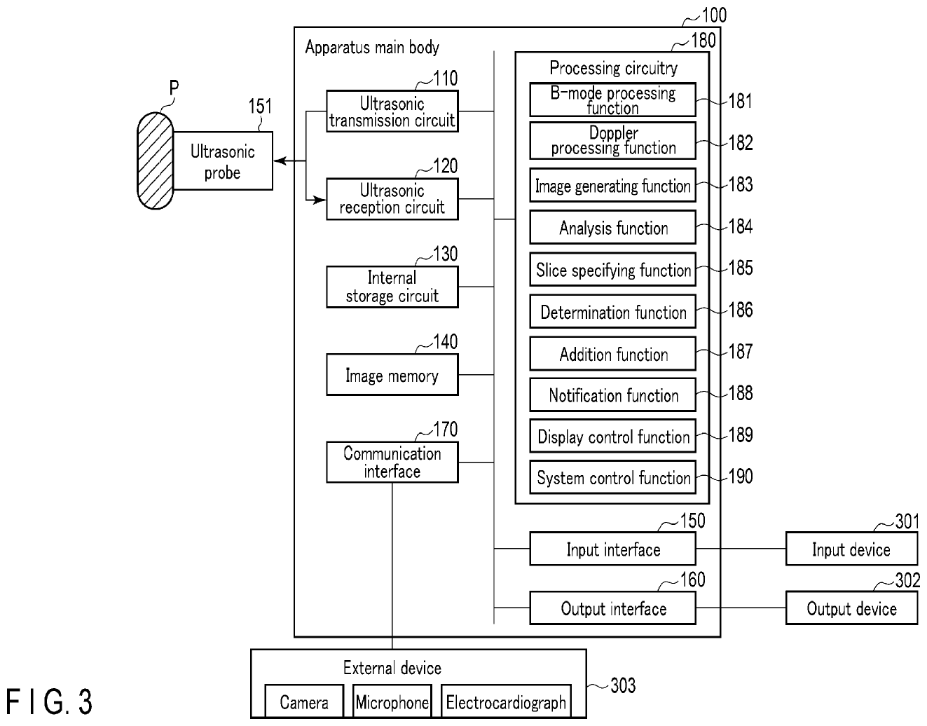

The analysis function 101 acquires and analyzes data such as camera images, voice data, and ECG (Electrocardiogram) waveforms from an external apparatus 303 in FIG. 3 (to be described later) and calculates an analysis value as an index for determining whether the data are images collected by the stress echocardiography method.

The slice specifying function 102 specifies imaged slices of a plurality of ultrasonic images.

The determination function 103 determines whether a plurality of ultrasonic images are images collected by the stress echocardiography method. For example, based on the type of slices of ultrasonic images and the situation in which the ultrasonic images are collected, the determination function 103 determines whether the ultrasonic images are images obtained by imaging based on the stress echocardiography method.

If the determination function 103 determines that ultrasonic images are images collected by the stress echocardiography examination method, the addition function 104 adds attribute information concerning at least one of the state of the subject and a slice of an ultrasonic image to each ultrasonic image.

The notification function 105 inquires the user whether ultrasonic images are collected by the stress echocardiography method.

The display control function 106 displays ultrasonic images and attribute information added to the ultrasonic images in association with each other and also displays a user interface that accepts editing from the user. If the determination function 103 determines that ultrasonic images are images collected by the stress echocardiography method, the display control function 106 activates application software used for a stress echocardiography examination.

Note that the present invention is not limited to a case in which each of the functions 101 to 106 is implemented by a single processing circuit. processing circuitry may be formed by combining a plurality of independent processors, and each of the functions 101 to 106 may be implemented by causing each processor to execute a program. Alternatively, each of the functions 101 to 105 may be stored as a program in the memory 11 or the like, and the function corresponding to each program may be implemented by causing the processing circuitry 10 to execute the program.

The memory 11 is a storage device storing various types of information, such as a ROM (Read Only Memory), a RAM (Random Access Memory), an HDD (Hard Disk Drive), an SSD (Solid State Device), or an integrated circuit storage device. In addition, the memory 11 may be a drive device that reads and writes various types of information with respect to portable storage media such as a CD-ROM drive, a DVD drive, and a flash memory. Note that the memory 11 need not always be implemented by a single storage device. For example, the memory 11 may be implemented by a plurality of storage devices. In addition, the memory 11 may be incorporated in another computer connected to the medical information processing apparatus 1 via a network.

The memory 11 stores a medical information processing program and the like according to this embodiment. Note that this program may be, for example, stored in advance in the memory 11. Alternatively, the program may be stored in non-transient storage media and distributed and may be read out from the non-transient storage media and installed in a memory 21.

The input interface 12 accepts various types of input operations from the user as a healthcare professional, converts the accepted input operations into electrical signals, and outputs the signals to the processing circuitry 10. In this embodiment, the input interface 12 according to this embodiment is connected to input devices such as a microphone, a mouse, a keyboard, a trackball, switches, buttons, a joystick, a touchpad, and a touch panel that inputs an instruction when the user touches the operation surface. In addition, the input devices connected to the input interface may be those installed in another computer connected via a network or the like.

The communication interface 13 performs data communication between medical image diagnostic apparatuses such as the image server 2, the electronic medical chart system 3, the medical information management application 4, and the ultrasonic diagnostic apparatus (not shown). The communication interface 13 performs data communication in compliance with, for example, preset known standards. For example, communication complying with HL7 is executed between the electronic medical chart system 3 and the medical information management application 4. In addition, the communication interface 13 executes communication complying with DICOM with the image server 2.

The display 14 displays various types of information in accordance with instructions from the processing circuitry 10. In addition, the display 14 may display a GUI (Graphical User Interface) or the like to accept various types of operations from the user. It is possible to use as needed, as the display 14, for example, an arbitrary display such as a CRT (Cathode Ray Tube) display, a liquid crystal display, an organic EL display, an LED display, or a plasma display. Note that the medical information processing apparatus 1 may not include the display 14, and a GUI may be displayed on an external display or may be displayed via a projector.

The ultrasonic diagnostic apparatus according to this embodiment will be described next with reference to the block diagram of FIG. 3.

The ultrasonic diagnostic apparatus shown in FIG. 3 includes an apparatus main body 100 and an ultrasonic probe 151. The apparatus main body 100 is an apparatus that generates an ultrasonic image based on a reflected wave signal received by the ultrasonic probe 151. The apparatus main body 100 includes an ultrasonic transmission circuit 110, an ultrasonic reception circuit 120, an internal storage circuit 130, an image memory 140, an input interface 150, an output interface 160, a communication interface 170, and processing circuitry 180.

Note that the ultrasonic diagnostic apparatus may be configured to incorporate the medical information processing apparatus 1. In this case, the same function as that of the medical information processing apparatus 1 may be implemented by using the internal storage circuit 130, the image memory 140, the input interface 150, the output interface 160, the communication interface 170, and the processing circuitry 180.

The ultrasonic transmission circuit 110 is a processor that supplies a driving signal to the ultrasonic probe 151. The ultrasonic transmission circuit 110 is implemented by, for example, a trigger generating circuit, a delay circuit, and a pulser circuit. The trigger generating circuit repeatedly generates rate pulses for the formation of transmission ultrasonic waves at a predetermined rate frequency. The delay circuit gives each rate pulse generated by the trigger generating circuit a delay time for each of a plurality of piezoelectric transducers which is necessary to focus an ultrasonic wave generated from the ultrasonic probe into a beam and determine transmission directivity. The pulser circuit applies a driving signal (driving pulse) to the plurality of ultrasonic transducers provided in the ultrasonic probe 151 at the timing based on the rate pulse. Changing the delay time given to each rate pulse using the delay circuit can arbitrarily adjust the transmission directions from the surfaces of a plurality of piezoelectric transducers.

The ultrasonic transmission circuit 110 can arbitrarily change the output intensity of ultrasonic waves by using a driving signal. The ultrasonic diagnostic apparatus can reduce the influence of the attenuation of ultrasonic waves in a patient P by increasing the output intensity. The ultrasonic diagnostic apparatus can acquire a reflected wave signal with a high S/N ratio at the time of reception by reducing the influence of the attenuation of ultrasonic waves.

In general, as ultrasonic waves propagate in the patient P, the intensity of vibrations (to be also referred to as acoustic power) of ultrasonic waves corresponding to the output intensity attenuates. The attenuation of acoustic power is caused by absorption, scattering, reflection, and the like. In addition, the degree of attenuation of acoustic power depends on the frequency of ultrasonic waves and the distances of ultrasonic waves in the radiation directions. For example, increasing the frequency of ultrasonic waves will increase the degree of attenuation. In addition, increasing the distances of ultrasonic waves in the radiation directions will increase the degree of attenuation.

The ultrasonic reception circuit 120 is a processor that generates a reception signal by performing various types of processing for a reflected wave signal received by the ultrasonic probe 151. The ultrasonic reception circuit 120 generates a reception signal with respect to the reflected wave signal of ultrasonic waves acquired by the ultrasonic probe 151. More specifically, the ultrasonic reception circuit 120 is implemented by, for example, a preamplifier, an A/D converter, a demodulator, and a beamformer (adder). The preamplifier performs gain correction processing by amplifying the reflected wave signals received by the ultrasonic probe 151 for each channel. The A/D converter converts a gain-corrected reflected wave signal into a digital signal. The demodulator demodulates the digital signal. The beamformer gives, for example, each demodulated digital signal a delay time required to determine reception directivity and adds a plurality of digital signals given with delay times. The beamformer generates a reception signal with an emphasized reflection component from a direction corresponding to the reception directivity by addition processing. Note that a reception signal may be called an IQ signal. In addition, the ultrasonic reception circuit 120 may cause the internal storage circuit 130 (to be described later) to store a reception signal (IQ signal) or may output the signal to the external apparatus 303 via the communication interface 170.

The internal storage circuit 130 has, for example, a storage medium that can be read by a processor, such as a magnetic storage medium, an optical storage medium, or a semiconductor memory. The internal storage circuit 130 stores a program for implementing ultrasonic transmission/reception, various types of data, and the like. The program and various types of data may be stored in advance in, for example, the internal storage circuit 130. Alternatively, the program and various types of data may be stored in, for example, a non-transient storage media and distributed and may be read out from the non-transient storage media and installed in the internal storage circuit 130. In addition, the internal storage circuit 130 stores B-mode image data, contrast-enhanced image data, image data concerning a blood flow video, and the like which are generated by the processing circuitry 180 in accordance with operations input from an input device 301 via the input interface 150. The internal storage circuit 130 can also transfer stored image data to the external apparatus 303 or the like via the communication interface 170. Note that the internal storage circuit 130 may store the reception signal (IQ signal) generated by the ultrasonic reception circuit 120 or transfer the signal to the external apparatus 303 or the like via the communication interface 170.

Note that the internal storage circuit 130 may be a drive device or the like that reads and writes various types of information with respect to portable storage media such as a CD drive, a DVD drive, and a flash memory. The internal storage circuit 130 can write stored data in a portable storage medium and cause the external apparatus 303 to store the data via the portable storage medium.

The image memory 140 has, for example, a storage medium that can be read by a processor, such as a magnetic storage medium, an optical storage medium, or a semiconductor memory. The image memory 140 saves image data corresponding to a plurality of frames immediately before a freeze operation input via the input interface 150. Image data stored in the image memory 140 is, for example, continuously displayed (cine-displayed).

The internal storage circuit 130 and the image memory 140 described above need not always be implemented by independent storage devices. The internal storage circuit 130 and the image memory 140 may be implemented by a single storage device. Alternatively, the internal storage circuit 130 and the image memory 140 each may be implemented by a plurality of storage devices.

The input interface 150 accepts various types of instructions from the operator via the input device 301. The input device 301 includes, for example, a mouse, a keyboard, panel switches, slider switches, a trackball, a rotary encoder, an operation panel, and a TCS (Touch Command Screen). The input interface 150 is connected to the processing circuitry 180 via, for example, a bus, converts an operation instruction input from the operator into an electrical signal, and outputs the electrical signal to the processing circuitry 180. Note that the input interface 150 is not limited to only those that are connected to physical operation components such as a mouse and a keyboard. Examples of the input interface include a circuit that accepts an electrical signal corresponding to an operation instruction input from an external input device provided independently of the medical information processing apparatus 1 and outputs the electrical signal to the processing circuitry 180.

The output interface 160 is an interface for outputting an electrical signal from the processing circuitry 180 to an output device 302. The output device 302 is an arbitrary display such as a liquid crystal display, an organic EL display, an LED display, a plasma display, or a CRT display. The output device 302 may be a touch panel display also serving as the input device 301. The output device 302 may further include a loudspeaker that outputs sound in addition to the display. The output interface 160 is connected to the processing circuitry 180 via, for example, a bus and outputs an electrical signal from the processing circuitry 180 to the output device 302.

The communication interface 170 is connected to the external apparatus 303 via, for example, a network and performs data communication with the external apparatus 303.

The processing circuitry 180 is a processor functioning as the main unit of the ultrasonic diagnostic apparatus. The processing circuitry 180 executes a program stored in the internal storage circuit 130 and implements a function corresponding to the program. The processing circuitry 180 includes, for example, a B-mode processing function 181, a Doppler processing function 182, an image generating function 183, an analysis function 184, a slice specifying function 185, a determination function 186, an addition function 187, a notification function 188, a display control function 189, and a system control function 190.

Note that since the analysis function 184, the slice specifying function 185, the determination function 186, the addition function 187, and the notification function 188 have functions similar to those of the medical information processing apparatus 1 shown in FIG. 2, a description of the functions will be omitted.

The B-mode processing function 181 is a function of generating B-mode data based on a reception signal received from the ultrasonic reception circuit 120. In the B-mode processing function 181, the processing circuitry 180 performs, for example, envelope detection processing, logarithmic processing, and the like for reception signals received from the ultrasonic reception circuit 120 to generate data (B-mode data) whose signal intensity is expressed by a luminance level. The generated B-mode data is stored as B-mode raw data on two-dimensional ultrasonic scanning lines (raster) in a raw data memory (not shown).

The Doppler processing function 182 is a function of generating data (Doppler information) by extracting motion information based on the Doppler effect of a moving object in an ROI (Region Of Interest) set in a scan region by performing frequency analysis of reception signals received from the ultrasonic reception circuit 120. The generated Doppler information is stored as Doppler raw data (to be also referred to as Doppler data) on two-dimensional ultrasonic scanning lines in the raw data memory (not shown).

More specifically, the processing circuitry 180 causes the Doppler processing function 182 to estimate, for example, as the motion information of the moving object, an average velocity, a mean variance value, an average power value, and the like at each of a plurality of sampling points and generate Doppler data representing the estimated motion information. The moving body is, for example, a blood flow, the tissue such as the cardia wall, or a contrast medium. The processing circuitry 180 according to the first embodiment causes the Doppler processing function 182 to estimate, as the motion information (blood flow information) of a blood flow, the average velocity of the blood flow, the variance value of blood flow velocities, the power value of a blood flow signal at each of a plurality of sampling points and generate Doppler data representing the estimated blood flow information.

The image generating function 183 is a function of generating B-mode image data based on the data generated by the B-mode processing function 181. For example, in the image generating function 183, the processing circuitry 180 generates image data for display (display image data) by converting (scan-converting) a scanning line signal sequence of ultrasonic scanning into a scanning line signal sequence in a video format represented by TV or the like. More specifically, the processing circuitry 180 generates two-dimensional B-mode image data (to be also referred to as ultrasonic image data) constituted by pixels by executing raw-pixel conversion for B-mode raw data stored in the raw data memory, for example, coordinate conversion corresponding to the ultrasonic scanning form employed by the ultrasonic probe 151. In other words, the processing circuitry 180 causes the image generating function 183 to generate a plurality of ultrasonic images (medical images) respectively corresponding to a plurality of consecutive frames from ultrasonic image data. In addition, the image generating function 183 may generate an optical image based on optically captured data.

In addition, the image generating function 183 generates Doppler image data with blood flow information being videoed by executing raw-pixel conversion with respect to Doppler raw data stored in the raw data memory. Doppler image data is average velocity image data, variance image data, power image data, or image data obtained by combining them. The image generating function 183 generates, as Doppler image data, color Doppler image data representing blood flow information in color and Doppler image data representing one piece of blood flow information in a wave shape based on grayscale. Color Doppler image data is generated at the time of executing the above blood flow video mode.

The display control function 189 is a function of causing the display as the output device 302 to display an image based on each of various types of ultrasonic image data generated by the image generating function 183. More specifically, for example, the processing circuitry 180 causes the display control function 189 to control display of the B-mode image data, the contrast-enhanced image data, or image data including both of them, generated by the image generating function 183, on the display. In addition, the display control function 189 controls display of optical images.

More specifically, the processing circuitry 180 causes the display control function 189 to generate display image data by converting (scan-converting) a scanning line signal sequence of ultrasonic scanning into a scanning line signal sequence in a video format represented by TV or the like. In addition, the processing circuitry 180 may execute various types of processing associated with a dynamic range, luminance (brightness), contrast, y curve correction, RGB conversion, and the like. The processing circuitry 180 may also add supplementary information such as character information of various types of parameters, scale marks, and body marks to display image data. Furthermore, the processing circuitry 180 may generate a GUI (Graphic User Interface) for allowing the operator to input various types of instructions with the input device and cause the display to display the GUI. Note that the display control function 189 implements a function similar to the display control function 106 of the medical information processing apparatus 1 shown in FIG. 2.

The system control function 190 is a function of comprehensively controlling the overall operation of the ultrasonic diagnostic apparatus. For example, the system control function 190 controls the ultrasonic transmission circuit 110 and the ultrasonic reception circuit 120 so as to execute ultrasonic scanning based on the transmission/reception conditions set by a setting function.

The external apparatus 303 is assumed to be a camera, a microphone, or an electrocardiograph. The camera images the whole body of the patient. The captured image data is stored in, for example, the image memory 140 via the communication interface 170. The microphone collects sound in an examination room as audio data. The collected audio data is stored in, for example, the internal storage circuit 130 via the communication interface 170. The electrocardiograph collects electrocardiographic information obtained from ECG electrodes connected to the patient P via a cable. The collected electrocardiographic information is stored in, for example, the internal storage circuit 130 via the communication interface 170.

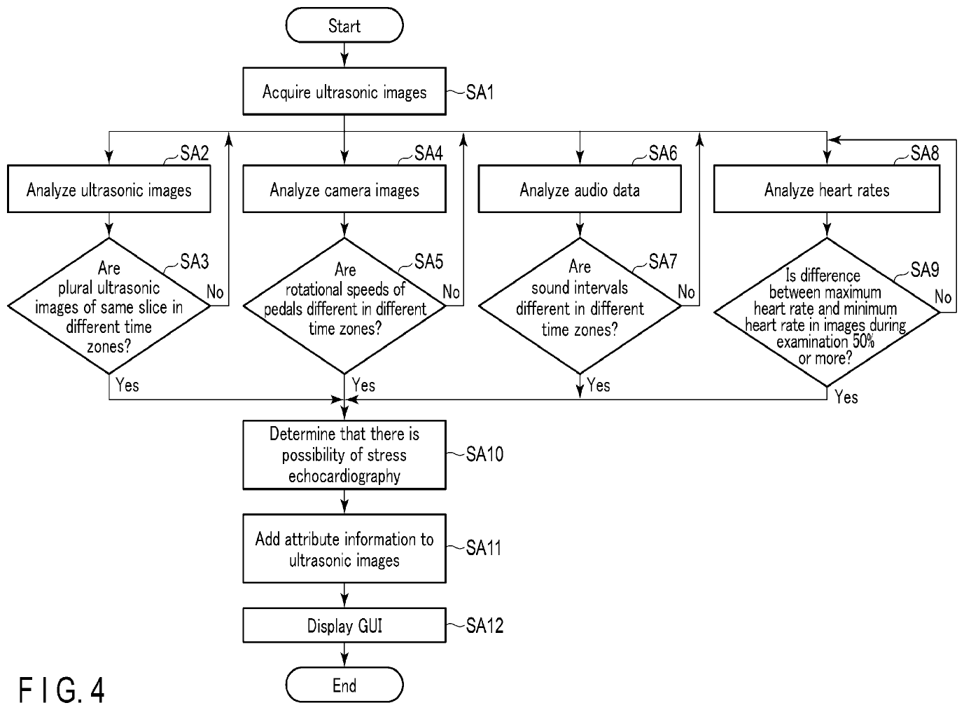

An example of the operation of the medical information processing apparatus 1 according to this embodiment will be described next with reference to the flowchart of FIG. 4. Although an example of the operation of the medical information processing apparatus 1 included in the ultrasonic diagnostic apparatus will be described below, the medical information processing apparatus 1 independent of the ultrasonic diagnostic apparatus can also execute the same operation. Assume a state in which the patient P is examined, starting by a general ultrasonic examination instead of a stress echocardiography examination, and a technician instructs the patient P in the middle of the examination to pedal a fitness bike so that stress is applied to the heart as the patient P pedals the fitness bike.

In step SA1, the processing circuitry 180 causes the slice specifying function 185 to acquire a plurality of ultrasonic images. More specifically, the processing circuitry 180 causes the image generating function 183 to generate a plurality of ultrasonic images along the time series based on the echo signals received by the ultrasonic probe 151 and causes the analysis function 184 to acquire the ultrasonic images.

In step SA2, the processing circuitry 180 causes the slice specifying function 185 to analyze each ultrasonic image and specify which slice of the imaging target region has been imaged to obtain the ultrasonic image. If, for example, the imaging target region is the heart, the types of slices are classified as four-chamber view, three-chamber view, two-chamber view, and left parasternal long axis view, and hence the slice specifying function 185 specifies to which slice the ultrasonic image corresponds. A slice specifying method may be configured to specify the types of slices by, for example, pattern matching based on image processing. Alternatively, this method may be configured to use an ultrasonic image as input data and estimate the type of slice by using a learned model obtained by training a machine learning model using the type of slice of the ultrasonic image as correct answer data (teaching label).

In step SA3, the processing circuitry 180 causes the determination function 186 to determine whether there are a plurality of ultrasonic images of the same slice in different time zones. If there are a plurality of ultrasonic images of the same slice in different time zones, the process advances to step SA10. If there are not a plurality of ultrasonic images of the same slice in different time zones, the process returns to step SA2 to repeat similar processing.

In step SA4, the processing circuitry 180 causes the analysis function 184 to analyze the camera images captured by the camera. In this case, the analysis function 184 calculates the rotational speed of the pedals of the fitness bike from camera images. For example, the analysis function 184 calculates the movement distance (rotational angle) of the pedals between frames of a given camera image and a camera image captured at the next imaging timing. If the patient P did not pedal, the movement distance of the pedals is zero. This indicates that the patient P is in a rest state (Rest). If the pedals have rotated, since the movement distance (or the rotational angle) of the pedals has changed, a rotational speed can be calculated from the movement distance (or the rotational angle).

In step SA5, the processing circuitry 180 causes the determination function 186 to determine whether a plurality of ultrasonic images captured in different time zones differ in the rotational speed of the pedals. In this case, if a plurality of ultrasonic images captured in different time zones, including an ultrasonic image obtained in an initial stage of the examination and an ultrasonic image captured, for example, a several minutes after the initial stage, differ in the rotational speed of the pedals, the process advances to step SA10. In contrast to this, if the rotational speed of the pedals obtained from the ultrasonic image in the initial stage of the examination is not different from the rotational speed of the pedals obtained from the ultrasonic image after the initial stage, the process returns to step SA4 to repeat similar processing.

In step SA6, the processing circuitry 180 causes the analysis function 184 to analyze audio data collected by the microphone. In this case, the analysis function 184 executes frequency spectrum analysis for the audio data to analyze at which pitch sound in a predetermined frequency band is generated.

In step SA7, the processing circuitry 180 causes the determination function 186 to determine whether sound intervals in different time zones are different. For example, in a stress echocardiography examination, a metronome is sometimes used to indicate the pace of motion of the patient P. Accordingly, frequency spectrum analysis may be performed to determine in which interval a frequency corresponding to the sound of the metronome is generated. If, therefore, the sound of the metronome is generated, the determination function 186 determines that the sound intervals are different in different time zones, and the process advances to step SA10. If the sound of the metronome is not generated, since the sound intervals are not different, the process returns to step SA6 to repeat similar processing.

In step SA8, the processing circuitry 180 causes the analysis function 184 to analyze the electrocardiographic data collected by the electrocardiograph. In this case, the analysis function 184 determines a heart rate from the electrocardiographic data.

In step SA9, the processing circuitry 180 causes the determination function 186 to determine whether the difference between the maximum value and the minimum value of the heart rate is 50% or more in the ultrasonic image during the examination. If the difference between the maximum value and the minimum value of the heart rate is 50% or more, the process advances to step SA10. If the difference is less than 50%, the process returns to step SA8 to repeat similar processing.

In step SA10, the processing circuitry 180 causes the determination function 186 to determine that there is a possibility that a stress echocardiography examination is executed.

In step SA11, the processing circuitry 180 causes the slice specifying function 185 and the addition function 187 to add attribute information to an ultrasonic image. The added attribute information includes, for example, a stress stage indicating which type and level of stress is applied and the type of slice. However, other types of information such as time information and patient information may be added.

In step SA12, the processing circuitry 180 causes the display control function 189 to display a GUI (Graphical User Interface) associating ultrasonic images with attribute information. The display control function 189 may automatically activate a stress echocardiography application instead of displaying a GUI. This makes it possible to refer to an imaging protocol for a stress echocardiography examination which is displayed by the stress echocardiography application.

Although step SA4 described above has exemplified the case in which the rotational speed of the pedals of the fitness bike is analyzed, the present invention is not limited to this, and the analysis function 184 may perform analysis by image analysis processing in accordance with the type of stress used in a stress echocardiography examination to determine which type of examination is executed. If, for example, a treadmill is used, the patient P exists on the treadmill in a camera image. In addition, if the leg portions of the patient P on the treadmill are moving, it is possible to determine that a stress echocardiography examination is been executed. Alternatively, whether the patient P is holding the handgrips may be determined by object detection processing or the like. If it is determined that the patient P is holding the handgrips, it may be determined that a stress echocardiography examination is being executed.

In step SA6, the processing circuitry 180 causes the analysis function 184 to perform audio recognition of audio data by existing audio recognition processing. If “pedal the fitness bike for 5 minutes” or “walk on the treadmill” is uttered, the processing circuitry 180 may cause the determination function 186 to determine that a stress echocardiography examination is being executed.

Note that steps SA2 and SA3, steps SA4 and SA5, steps SA6 and SA7, and steps SA8 and SA9 may be executed in a random order, and analysis may be started from any data. Alternatively, if one of the conditions in steps SA3, SA5, SA7, and SA9 is satisfied, it may be determined that there is a possibility that a stress echocardiography examination is being executed. In addition, if a plurality of conditions are satisfied, it may be determined that there is a possibility that a stress echocardiography examination is being executed.

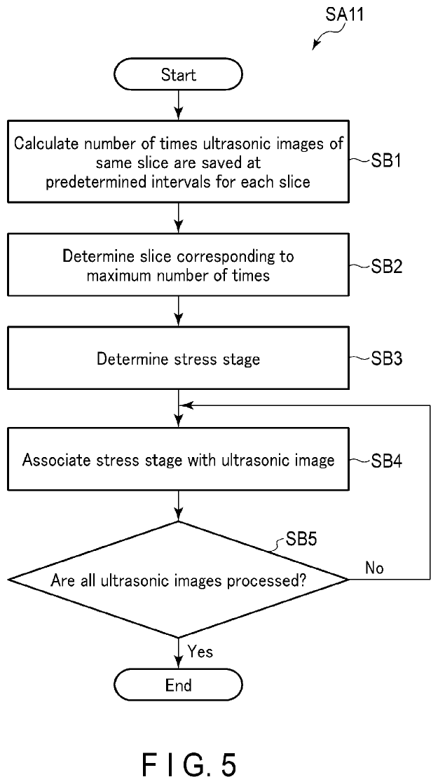

The details of attribute information addition processing concerning step SA11 in the medical information processing apparatus 1 according to this embodiment will be described next with reference to the flowchart of FIG. 5.

In step SB1, the processing circuitry 180 causes the slice specifying function 185 to acquire ultrasonic images collected in an examination and calculate the number of times an ultrasonic image of the same slice is saved at predetermined intervals for each slice. Assume a case in which the following images are saved in the same examination: “4ch (four-chamber view)”, “2ch (two-chamber view)”, “LV Inflow (left ventricular inflow)”, “Sep e′ (left ventricular diastolic function)”, “LVOT (left ventricular outflow tract)”, “4D”, “Unknown (not classified)”, “4ch”, “2ch”, “LV Inflow”, “Sep e′”, “LVOT”, “4D”, “RV Strain (right ventricular strain)”, “TAPSE (tricuspid annular plane systolic excursion)”, and “4ch”. In this case, since the slice “4ch” is acquired three times, and “2ch”, “LV Inflow”, “Sep e′”, and “LVOT” each are acquired two times, ultrasonic images having these slices are calculation targets.

In step SB2, the processing circuitry 180 causes the slice specifying function 185 to determine a slice that is calculated the maximum number of times in step SB1. In the case of step SB1, since a slice of “4ch” is calculated three times, that is, the maximum number of times, it is determined that the slice calculated the maximum number of times is “4ch”.

In step SB3, the processing circuitry 180 causes the determination function 186 to determine the stress stages of ultrasonic images with reference to the maximum slice and classify the ultrasonic images for each stress stage. More specifically, the stress stage of the first ultrasonic image with the slice type “4ch” may be classified as “rest state (Rest)”, the stress stage of the second ultrasonic image may be classified as “stress 1 (Post 1)”, and the stress stage of the third ultrasonic image may be classified as “stress 2 (Post 3)”. Note that stress stages may be classified as types such as “pre-stress”, “during-stress”, and “post-stress”.

In step SB4, the processing circuitry 180 causes the addition function 187 to determine, for each determined stress stage, images saved within a predetermined time from the ultrasonic images included in the stress stage as ultrasonic images in the same stress stage. The predetermined time is, for example, one minute, but the user may set a desired value. If there are a plurality of images correspond to a plurality of stress stages, an image with a shorter time difference may be selected.

In step SB5, the processing circuitry 180 causes the determination function 186 to determine whether all the ultrasonic images obtained in the examination have been processed. If all the ultrasonic images have been processed, the processing in step SA11 is terminated. If there is any unprocessed ultrasonic image, the process returns to step SB4 to repeat similar processing.

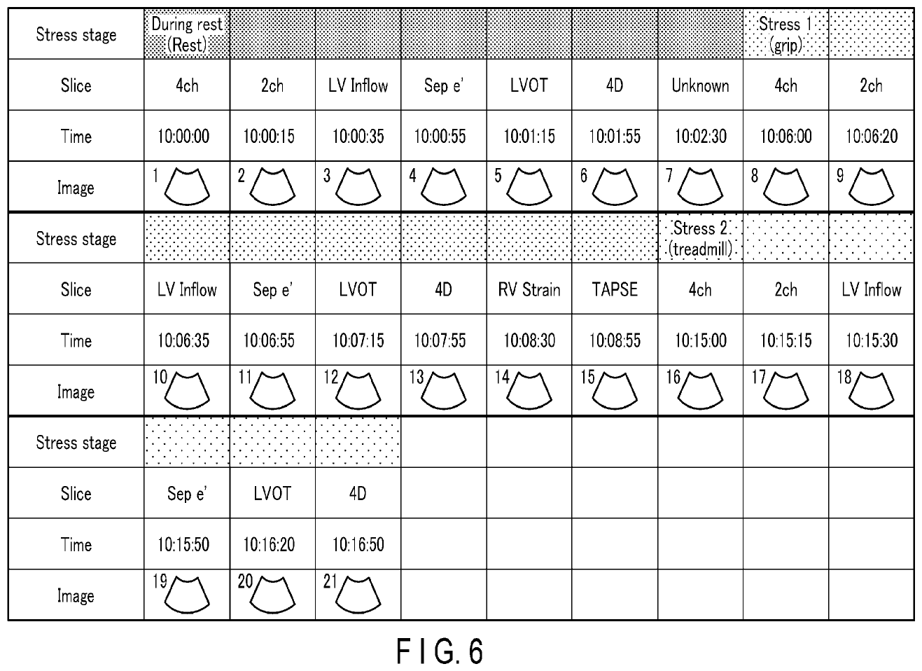

An example of a GUI associating ultrasonic images with attribute information according to this embodiment will be described next with reference to FIG. 6.

FIG. 6 shows a GUI associating ultrasonic images with attribute information. As the attribute information, stress stages (phase), slices (View), and times are associated with ultrasonic images. More specifically, in the example shown in FIG. 6, the stress stage “Rest”, the slice “4ch”, the time “10:00:00”, and the image “ultrasonic image 1” are associated with each other. Note that stress types may be displayed in the items of stress stages. In this case, displaying stress types such as “grip” as handgrip and “treadmill” allows the user to easily discriminate an ultrasonic image in a state in which a specific type of stress is applied to the user.

In this case, ultrasonic image 1 to ultrasonic image 7 fall within one minute from the first ultrasonic image, and the time difference between ultrasonic image 7 and ultrasonic image 8 is larger than one minute. Likewise, ultrasonic image 8 to ultrasonic image 15 and ultrasonic image 16 to ultrasonic image 21 fall within one minute. The time difference between ultrasonic image 15 and ultrasonic image 16 is larger than one minute. Accordingly, seeing the GUI shown in FIG. 6 allows the user to immediately understand that ultrasonic image 1 to ultrasonic image 7 are determined to belong to the stress stage of the patient P different from that to which ultrasonic image 8 and the subsequent ultrasonic images belong.

In addition, the above information may be displayed together with information such as a camera image or moving image captured in the examination room which serves as a material for the determination of a stress stage, audio data in the examination room, and a heart rate.

Note that the user may be allowed to perform editing such as changing, adding, and deleting a stress stage, attribute information such as a slice, and an ultrasonic image or adding a mark via the input device 301.

Note that in the above case, the ultrasonic diagnostic apparatus (medical information processing apparatus 1) determines from various types of information concerning the situation in the examination room whether a stress echocardiography examination is being executed. However, an inquiry may be provided to the user to acquire information indicating whether a stress echocardiography examination is being executed in accordance with a user instruction.

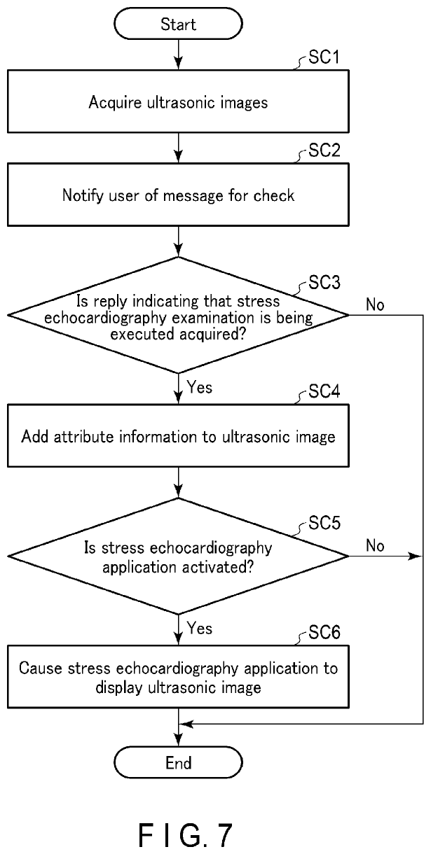

A case in which execution/non-execution of a stress echocardiography examination is determined in accordance with a user instruction will be described with reference to the flowchart of FIG. 7.

In step SC1, as in step SA1, the processing circuitry 180 causes the slice specifying function 185 to acquire an ultrasonic image. Subsequently, for example, whether a stress echocardiography examination has been executed is determined by any processing in step SA2 to step SA9 shown in FIG. 4. Assume that in this case, the processing circuitry 180 causes the determination function 186 to determine that a stress echocardiography examination has been executed in step SC5.

In step SC2, the processing circuitry 180 causes the notification function 188 to output a message for checking whether a stress echocardiography examination has been executed to the output device 302 via, for example, the output interface 160. For example, the processing circuitry 180 may cause the notification function 188 and the display control function 189 to display the message in a popup window together with the “Yes” and “No” buttons. The user may select either of the “Yes” and “No” buttons.

In step SC3, the processing circuitry 180 causes the determination function 186 to determine whether or not a replay indicating that a stress echocardiography examination is being executed is acquired. For example, selecting the “Yes” button makes it possible to determine that a reply indicating that a stress echocardiography examination is being executed is acquired. If a reply indicating that a stress echocardiography examination is being executed is acquired, the process advances to step SC4. If a reply indicating that a stress echocardiography examination is not being executed is acquired, that is, the “No” button is selected, the processing is terminated.

In step SC4, since a stress echocardiography examination is being executed, the processing circuitry 180 causes the addition function 187 to add attribute information to the ultrasonic image as in the case of step SA11.

In step SC5, the processing circuitry 180 causes the determination function 186 to determine whether to activate the stress echocardiography application. For example, the determination function 186 may notify the user of a message like “Do you activate the stress echocardiography application?”. In this case, if the “Yes” button is selected, the determination function 186 may determine to activate the stress echocardiography application. If the stress echocardiography application is to be activated, the process advances to step SC6. If the stress echocardiography application is not to be activated, the processing is terminated.

In step SC6, the processing circuitry 180 causes the display control function 189 to activate the stress echocardiography application. The stress echocardiography application then imports and displays an ultrasonic image to which attribute information is added in step SC4.

Note that the description about step SC5 has exemplified the case in which the user checks whether to activate the stress echocardiography application. However, the present invention is not limited to this, and the stress echocardiography application may be automatically activated without any inquiry to the user.

Step SC2 described above has exemplified the case in which a message for checking whether a stress echocardiography examination is being executed is output during an examination by the ultrasonic diagnostic apparatus. However, at the timing of outputting a notification of the end of an examination after the acquisition of a series of examination images, a message for checking whether the executed examination is a stress echocardiography examination may be displayed. This makes it possible to execute the determination of a stress echocardiography examination without hindering the examination by displaying a check message during the examination.

In addition, the above embodiment has exemplified the case in which it is determined in real time whether a stress echocardiography examination is being executed during an examination. However, the processing by the medical information processing apparatus can be equally applied to any past ultrasonic images for which various types of information in the examination room have been acquired by the external apparatus 303.

According to the embodiment described above, it is determined from various types of information such as a camera image and audio data in the examination room whether a stress echocardiography examination has been executed, and attribute information is added to each ultrasonic image in a series of examinations as determination targets if a stress echocardiography examination has been executed. This makes it possible to add attribute information to even a past ultrasonic image in a rest state to which attribute information cannot be tagged in general unless the stress echocardiography application is activated in advance. Accordingly, ultrasonic images can be used in the stress echocardiography application as postprocessing. This can improve the convenience.

The term “processor” means a circuit such as a CPU (Central Processing Unit), a GPU (Graphics Processing Unit), ASIC (Application Specific Integrated Circuit), or a programmable logic device (for example, an SPLD (Simple Programmable Logic Device), a CPLD (Complex Programmable Logic Device), or an FPGA (Field Programmable Logic Device)). When the processor is, for example, a CPU, the processor reads out a program stored in a storage circuit and executes the program to implement the corresponding function. In contrast to this, if the processor is, for example, an ASIC, the corresponding function is directly incorporated as a logic circuit in the circuit of the processor instead of saving a program in the storage circuit. Note that each processor according to this embodiment may be formed as one processor by combining a plurality of independent circuits to implement the corresponding function as well as being formed as a single circuit. In addition, a plurality of constituent elements in FIG. 1 may be integrated into one processor to implement the corresponding function.

In addition, each function according to this embodiment can also be implemented by installing programs for executing the corresponding processing in a computer such as a workstation and expanding them in a memory. In this case, the programs which can cause the computer to execute the corresponding techniques can be distributed by being stored in storage media such as magnetic disks (hard disks and the like), optical disks (CD-ROMs, DVDs, and the like), and semiconductor memories.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing apparatus comprising processing circuitry configured to:
determine whether or not a plurality of ultrasonic images are images collected by a stress echocardiography method; and
add attribute information to each of the ultrasonic images if it is determined that the ultrasonic images are images collected by the stress echocardiography method, the attribute information concerning at least one of a stress state of a subject and a slice of the ultrasonic image.

2. The medical information processing apparatus according to claim 1, wherein the processing circuitry determines, based on a type of slices of the ultrasonic images and a situation in which the ultrasonic images are collected, whether or not the ultrasonic images are images acquired by the stress echocardiography method.

3. The medical information processing apparatus according to claim 1, wherein the stress state of the subject includes information indicating one of a pre-stress state, a during-stress state, and a post-stress state.

4. The medical information processing apparatus according to claim 3, wherein the attribute information further includes information concerning a type of stress.

5. The medical information processing apparatus according to claim 1, wherein the processing circuitry determines, based on at least one of the type of slices of the ultrasonic images, a camera image in an examination room, audio data acquired in the examination room, and information of a heart rate of the subject when the ultrasonic images are acquired, whether or not the ultrasonic images are images collected by the stress echocardiography method.

6. The medical information processing apparatus according to claim 1, wherein the processing circuitry is further configured to:
notify a user of an inquiry whether or not ultrasonic images are collected by the stress echocardiography method; and
determine that an examination is being executed by the stress echocardiography method if the user has replied that the ultrasonic images have been collected by the stress echocardiography method.

7. The medical information processing apparatus according to claim 1, the processing circuitry is further configured to display the ultrasonic images and the attribute information in association with each other and display a user interface configured to accept editing from the user.

8. The medical information processing apparatus according to claim 1, the processing circuitry is further configured to activate application software used for a stress echocardiography examination if it is determined that the ultrasonic images are images collected by the stress echocardiography method.

9. The medical information processing apparatus according to claim 8, wherein the processing circuitry imports and display an ultrasonic image to which the attribute information is added.

10. A non-transitory computer readable medium including computer executable instructions, wherein the instructions, when executed by a processor, cause the processor to perform a method comprising:
determining whether or not a plurality of ultrasonic images are images collected by a stress echocardiography method; and
adding attribute information to each of the ultrasonic images if it is determined that the ultrasonic images are images collected by the stress echocardiography method, the attribute information concerning at least one of a stress state of a subject and a slice of the ultrasonic image.

11. An ultrasonic diagnostic apparatus comprising:
an ultrasonic probe; and
processing circuitry configured to:
generate ultrasonic images by using echo signals received by the ultrasonic probe;
determine whether or not the ultrasonic images are images collected by a stress echocardiography method; and
add attribute information to each of the ultrasonic images if it is determined that the ultrasonic images are images collected by the stress echocardiography method, the attribute information concerning at least one of a stress state of a subject and a slice of the ultrasonic image.
